(19) United States
(12) Patent Application Publication
(10) Pub. No.: US 2025/0214914 A1
(43) Pub. Date: Jul. 3, 2025

(54) METHOD FOR PRODUCING AROMATICS VIA LIGHT HYDROCARBONS FROM METHANE CONTAINING FEED

(71) Applicant: Sogang University Research & Business Development Foundation, Seoul (KR)

(72) Inventors: Kyoung-Su HA, Seoul (KR); Hee Hwan Kim, Seoul (KR); JongHyun Jeon, Seoul (KR)

(21) Appl. No.: 19/003,680

(22) Filed: Dec. 27, 2024

(30) Foreign Application Priority Data
Dec. 29, 2023 (KR) 10-2023-0197499
Dec. 6, 2024 (KR) 10-2024-0180531

(51) Int. Cl. C07C 2/46; B01J 8/06; B01J 29/40; B01J 37/08; C01B 39/02; C07C 2/80

(52) U.S. Cl. CPC C07C 2/46; B01J 8/065; B01J 8/067; B01J 29/40; B01J 37/08; C01B 39/026; C07C 2/80; B01J 2208/00504; C07C 2529/06

(57) ABSTRACT

Disclosed is a process carried out by comprising: a first reaction unit for converting a methane-containing feed into light hydrocarbons in a low-temperature plasma reaction; a separator for selectively separating/removing a portion of the mixture; and a second reaction unit for converting the light hydrocarbons passing through the first reaction unit or the separator into aromatics on high-temperature zeolite and, if needed, further comprising: a pre-treatment unit for treating the feed; and a post-treatment unit for separating and purifying the products of the second reaction unit.

### CROSS-REFERENCE TO RELATED APPLICATIONS

This application claims benefit of Korean Patent Application Nos. 10-2023-0197499, filed on 29 Dec. 2023 and 10-2024-0180531, filed on 6 Dec. 2024. The entire disclosures of the applications identified in this paragraph are incorporated herein by references.

### FIELD

The present disclosure was also made with the support of the Ministry of Science and ICT of the Republic of Korea, under Sub-project No. 2018M3D3A1A0101800432 within Project Identification No. 1055001136, which was conducted in the research project named “Development of Catalyst Technology for Methane-To-Aromatics Via Light Hydrocarbons” in the research program titled “Climate Change Mitigation Technologies”, by Sogang University, under the management of the National Research Foundation of Korea, from 1 Jan. 2023 to 29 Feb. 2024.

The present disclosure was also made with the support of the Ministry of Science and ICT of the Republic of Korea, under Sub-project No. 00466477 within Project Identification No. 2710018537, which was conducted in the research project named “Development of Technology for High-Concentration C1 Chemical Species from Off-Gas for Lactone and Organic Acid Production” in the research program titled “Global C1 Gas Refinery Value Up (R&D)”, by Chung-Ang University, under the management of the National Research Foundation of Korea, from 1 Jul. 2024 to 31 Dec. 2024.

This application claims priority to and the benefit of Korean Patent Application No. 10-2023-0197499 filed in the Korean Intellectual Property Office on 29 Dec. 2023 and Korean Patent Application No. 10-2024-0180531 filed in the Korean Intellectual Property Office on 6 Dec. 2024, the disclosures of which are incorporated herein by reference.

The present disclosure relates to a method for producing aromatics from a methane-containing feed via light hydrocarbons and, specifically, to a process for producing high value-added aromatics from a methane-containing carbon compound feed, such as shale gas, natural gas, coal bed methane, by-product gas, and waste gas, by using non-equilibrium low-temperature plasma discharge conversion and zeolite-based high-temperature conversion.

### BACKGROUND

High value-added aromatics are meant to compounds, such as benzene, toluene, xylenes, ethylbenzene, and styrene. These compounds are monomers required for polymer production or precursors for preparing monomers, and are manufactured mainly by thermal cracking or fluidized catalytic cracking (FCC) of naphtha. However, many attempts have been made to manufacture aromatics, which are petrochemical compounds, from resources (coal, natural gas, etc.) that can replace petroleum, due to the recent depletion of petroleum resources and high oil prices.

Meanwhile, shale gas, which has recently received attention, is usually composed of CH₄ (80-90% v/v) and other light alkanes (e.g., C₂H₆, C₃H₈), and various attempts have been made to produce light olefins from shale gas, like in typical petroleum resources.

In current processes that are generally employed, aromatics are produced during thermal cracking and fluidized catalytic cracking of naphtha obtained from petroleum. Out of these, BTX (benzene, toluene, and xylenes components) are very important aromatics, wherein benzene is used as a raw material for styrene monomer, nylon, cumene, or the like, and toluene is often converted to benzene through a conversion facility due to a relatively low added value thereof. Paraxylene is used for a polyester film or fiber.

The production pathways of major aromatics primarily rely on petroleum. Existing petrochemical methods have difficulties in that the additional induction of chemical conversion of naphthene or the additional conversion from olefins need to be conducted in order to further increase yields or productivity of aromatics.

Therefore, much research and development is still needed with respect to techniques for producing aromatics from petroleum replacements. There is an urgent need to develop manufacturing techniques that enable the use of by-product gas and waste gas due to environmental considerations as well as the conversion utilizing shale gas, natural gas, and the like in terms of efficient resource utilization.

### SUMMARY OF THE INVENTION

The present inventors identified that aromatics can be produced from various hydrocarbon feeds containing a methane component by performing a low-temperature plasma reaction process and a zeolite-based high-temperature reaction process.

Accordingly, an aspect of the present disclosure is to provide a method for producing aromatics from a methane-containing mixture, the method including:
a first reaction step of converting a methane-containing mixture, which is supplied, into C2 to C4 light hydrocarbons by inducing a low-temperature plasma reaction in the methane-containing mixture; and
a second reaction step of synthesizing aromatics from a substrate containing the products in the first reaction step, in the presence of a zeolite catalyst.

Another aspect of the present disclosure is to provide an apparatus for producing aromatics from a methane-containing mixture, the apparatus including:
a first reaction unit including a low-temperature plasma reactor including a reaction tube where a low-temperature plasma is generated; and
a second reaction unit including a zeolite-based high-temperature reactor: including a reaction tube having a zeolite catalyst bed therein; and a furnace adjacent to the reaction tube.

The present disclosure is directed to a method for producing aromatics from a methane-containing feed via light hydrocarbons, and it was validated that the method according to the present disclosure can produce aromatics from various hydrocarbon feeds containing a methane component by performing a low-temperature plasma process and a zeolite-based high-temperature reaction process.

In accordance with an aspect of the present disclosure, there is provided a method for producing aromatics from a methane-containing mixture, the method including:
a first reaction step of converting a methane-containing mixture, which is supplied, into C2 to C4 light hydrocarbons by inducing a low-temperature plasma reaction in the methane-containing mixture; and
a second reaction step of synthesizing aromatics from a substrate containing the products in the first reaction step, in the presence of a zeolite catalyst.

In the present disclosure, the first reaction step may be performed by generating plasma through the supply of an alternating current power at an applied voltage of 10-20 kV and a frequency of 0.1 to 5 kHz.

The applied voltage may be preferably 10 to 18 kV, 10 to 16 kV, 12 to 20 kV, 12 to 18 kV, 12 to 16 kV, 14 to 20 kV, or 14 to 18 kV, for example, 14 to 16 kV, but is not limited thereto.

The frequency may be 0.1 to 3 kHz, 0.1 to 2 kHz, 0.1 to 1 kHz, 0.2 to 5 kHz, 0.2 to 3 kHz, 0.2 to 2 kHz, 0.2 to 1 kHz, 0.5 to 5 kHz, 0.5 to 3 kHz, 0.5 to 2 kHz, 0.5 to 1 kHz, 1 to 5 kHz, or 1 to 3 kHz, for example, 1 to 2 kHz, but is not limited thereto.

In the present disclosure, the first reaction step may be performed in a temperature condition of 25 to 100° C., but is not limited thereto.

In the separation step, a portion or the entirety of the unreacted methane-containing mixture may be separated for recycling into the first reaction step, or a portion of the produced light hydrocarbons may be separated or removed.

The method may further include a pre-treatment step of controlling the methane-containing mixture before the methane-containing mixture is supplied to the first reaction step.

In the present disclosure, the methane-containing mixture may contain methane and nitrogen at a volume ratio of 8:2 to 2:8, preferably a volume ratio of 8:2 to 3:7, 8:2 to 4:6, 8:2 to 5:5, 7:3 to 2:8, 7:3 to 3:7, 7:3 to 4:6, 7:3 to 5:5, 6:4 to 2:8, 6:4 to 3:7, 6:4 to 4:6, 6:4 to 5:5, 5:5 to 2:8, or 5:5 to 3:7, and for example, a volume ratio of 5:5 to 4:6, but is not limited thereto.

The first reaction step may be performed for 100 to 2,000 minutes while a reactant is supplied at a flow rate of 20 to 60 sccm.

The flow rate may be preferably 20 to 50 sccm, 20 to 40 sccm, 30 to 60 sccm, 30 to 50 sccm, 30 to 40 sccm, or 40 to 60 sccm, for example, 40 to 50 sccm, but is not limited thereto.

The reaction time may be preferably 100 to 1,500 minutes, 100 to 1,200 minutes, 100 to 1,000 minutes, 500 to 2,000 minutes, 500 to 1,500 minutes, 500 to 1,200 minutes, 500 to 1,000 minutes, 800 to 2,000 minutes, 800 to 1,500 minutes, 800 to 1,200 minutes, 800 to 1,000 minutes, 1,000 minutes to 2,000 minutes, or 1,000 minutes to 1,500 minutes, and for example, 1,000 minutes to 1,200 minutes, but is not limited thereto.

In the present disclosure, the first reaction step may be performed in the presence of a catalyst with a particle size of 0 to 100 μm.

The particle size may be preferably 1 to 100 μm, 10 to 100 μm, or 20 to 100 μm, and for example, 50 to 100 μm, but is not limited thereto.

The catalyst in the first reaction step may be used wherein a catalyst compound is provided by a combination of at least one selected from a dielectric material group consisting of TiO₂, MgO, Al₂O₃, amorphous silica, and crystalline silica, and a metal material effective for a dehydrogenation or coupling reaction, such as nickel (Ni), ruthenium (Ru), or platinum (Pt), is supported thereon. The catalyst compound promotes the generation of radicals from the methane feed and the production of light hydrocarbons through a coupling reaction between the radicals, and a reactor filled with the catalyst compound is provided.

In the present disclosure, the method may further include a separation step of adjusting the amount or composition of a reactant, which is generated in the first reaction step and supplied for performing the second reaction step.

In an embodiment of the present disclosure, a light hydrocarbon mixture synthesized in the low-temperature plasma reactor is separated/purified, either entirely or partially, and subjected to a conversion reaction in a second reactor. In the second reactor, the solid acid catalyst zeolite is used for a catalyst bed, and the reaction temperature is in the range of 500 to 800° C. Various zeolites, such as ZSM-5, MCM-22, X, Y, and A, are usable, and H-form zeolite is used to utilize acid sites. In the synthesis of zeolite, Na ion-form zeolite is mainly synthesized, and this zeolite is converted into an ammonium ion-form (NH4⁺-form) by ion exchange with a precursor solution of ammonium ions, and then burned at an appropriate temperature, thereby obtaining desired H-form zeolite.

In the present disclosure, the second reaction step may be performed by using C₂ to C₄ light hydrocarbons, nitrogen, and hydrogen as a substrate.

The substrate may be a mixture gas containing 2 to 15 mol % C₂H₂, 0.2 to 8 mol % C₂H₄, 0.2 to 8 mol % C₂H₆, 0.2 to 8 mol % C3 hydrocarbon, 0.5 to 12 mol % C4 hydrocarbon, 40 to 60 mol % N₂, and 30 to 45 mol % H₂, and the mixture gas may contain preferably 5 to 10 mol % C₂H₂, 0.5 to 4 mol % C₂H₄, 0.5 to 4 mol % C₂H₆, 0.5 to 4 mol % C3 hydrocarbon, 1 to 6 mol % C4 hydrocarbon, 45 to 55 mol % N₂, and 33 to 42 mol % H₂ hydrocarbon, for example, 7 to 8 mol % C₂H₂, 1 to 2 mol % C₂H₄, 1 to 2 mol % C₂H₆, 1 to 2 mol % C3 hydrocarbon, 2 to 3 mol % C4 hydrocarbon, 48 to 52 mol % N₂, and 35 to 40 mol % H₂, but is not limited thereto.

In the present disclosure, the second reaction step may be performed under a temperature condition of 500 to 800° C. and a space velocity of 10,000 to 30,000 mL/g_{cat}⁻¹h⁻¹.

The temperature condition may be 500 to 750° C., 500 to 700° C., 600 to 800° C., 600 to 750° C., 600 to 700° C., 650 to 800° C., 650 to 750° C., 650 to 700° C., or 700 to 800° C., for example, 700 to 750° C., but is not limited thereto.

The space velocity may be preferably 10,000 to 25,000 mL/g_{cat}⁻¹h⁻¹, 10,000 to 22,000 mL/g_{cat}⁻¹h⁻¹, 10,000 to 20,000 mL/g_{cat}⁻¹h⁻¹, 15,000 to 25,000 mL/g_{cat}⁻¹h⁻¹, 15,000 to 22,000 mL/g_{cat}⁻¹h⁻¹, 15,000 to 20,000 mL/g_{cat}⁻¹h⁻¹, 18,000 to 25,000 mL/g_{cat}⁻¹h⁻¹, 18,000 to 22,000 mL/g_{cat}⁻¹h⁻¹, 18,000 to 20,000 mL/g_{cat}⁻¹h⁻¹, or 20,000 to 25,000 mL/g_{cat}⁻¹h⁻¹, for example, 20,000 to 22,000 mL/g_{cat}⁻¹h⁻¹, but is not limited thereto.

In the present disclosure, the zeolite catalyst may have a Si/Al ratio of 6 to 17, preferably a Si/Al ratio of 6 to 15, 6 to 13, 6 to 12, 9 to 17, 9 to 15, 9 to 13, 9 to 12, 11 to 17, 11 to 15, or 11 to 13, for example, a Si/Al ratio of 11 to 12, but is not limited thereto.

In the present disclosure, the aromatics may be one or more selected from the group consisting of benzene, toluene, xylenes, ethyl benzene, and styrene, but is not limited thereto.

The method may further include a post-treatment of separating or purifying the products in the second reaction step into a target substance.

In an embodiment of the present disclosure, the method for producing aromatics from a methane-containing mixture, the method including:
a pre-treatment step of pre-treating/separating/purifying a methane-containing hydrocarbon feed;
a first reaction step of, after the pre-treatment, introducing the methane-concentrated mixture into a low-temperature plasma reactor to convert the mixture into a light hydrocarbon mixture;
a separation step of selectively separating/purifying a portion of the light hydrocarbon mixture; and
a second reaction step of aromatizing the light hydrocarbon mixture in a zeolite-based high-temperature reactor.

In accordance another aspect of the present disclosure, there is provided an apparatus for producing aromatics from a methane-containing mixture, the apparatus including:
a first reaction unit including a low-temperature plasma reactor including a reaction tube where a low-temperature plasma is generated; and
a second reaction unit including a zeolite-based high-temperature reactor including: a reaction tube having a zeolite catalyst bed therein; and a furnace adjacent to the reaction tube.

The low-temperature plasma reactor may further include a high-voltage (HV) electrode inside the reaction tube, and a ground electrode and a capacitor outside the reaction tube.

The low-temperature plasma reactor may be a dielectric barrier discharge plasma reactor, but is not limited thereto.

The high-voltage electrode may be a metal rod with a diameter of 1 to 5 mm, preferably, a metal rod with a diameter of 1 to 4 mm, 1 to 3 mm, 2 to 5 mm, 2 to 4 mm, 2 to 3 mm, or 3 to 5 mm, for example, a metal rod with a diameter of 3 to 4 mm, but is not limited thereto.

The metal rod of the high-voltage electrode may be a stainless steel rod.

The ground electrode may be in the form in which a metal wire is wound on an outer wall of the reaction tube in an area of 80 to 300 mm in length, preferably in an area of 80 to 240 mm, 80 to 180 mm, 80 to 160 mm, 120 to 300 mm, 120 to 240 mm, 120 to 180 mm, 120 to 160 mm, 150 to 300 mm, 150 to 240 mm, or 150 to 180 mm, and for example, in an area of 150 to 160 mm in length, but is not limited thereto.

The metal wire of the ground electrode may be a stainless steel wire.

In the present disclosure, the low-temperature plasma reactor may be a multi-tubular reactor having two or more reaction tubes.

The reaction tube where low-temperature plasma is generated may be composed of an alumina material with an inner diameter of 3 to 10 mm and a thickness of 1 to 5 mm.

The inner diameter of the reaction tube may be 3 to 8 mm, 3 to 6 mm, 5 to 10 mm, 5 to 8 mm, 5 to 6 mm, or 6 to 10 mm, for example, 6 to 8 mm, but is not limited thereto.

The thickness of the reaction tube may be 1 to 4 mm, 1 to 3 mm, 1 to 2 mm, 2 to 5 mm, or 2 to 4 mm, for example, 2 to 3 mm, but is not limited thereto.

Both ends of the reaction tube where low-temperature plasma is generated may independently include mixing sections capable of inducing the charging or mixing of a substance.

In the present disclosure, the first reaction unit may further include a transformer, an AC power supply, and an oscilloscope analyzer.

In the present disclosure, the apparatus may further include a separator configured to adjust the amount or composition of an inflowing material, which is generated in the first reaction step and supplied for performing the second reaction step.

In the present disclosure, the apparatus may further include a pre-treatment unit configured to control the inflow of a reactant into the first reaction unit.

In the present disclosure, the second reaction unit may further include a mass flow controller (MFC), an on-line gas chromatography (GC), a thermal conductivity detector (TCD), and a thermal flame ionization detector (FID).

In the present disclosure, the apparatus may further include a post-treatment unit configured to separate or purify a target substance from the product in the second reaction unit.

In the present disclosure, the reaction tube including the zeolite catalyst bed therein may be a quartz material with an inner diameter of 5 to 15 mm and a length of 270 to 810 mm.

The inner diameter of the reaction tube may be 5 to 12 mm, 5 to 10 mm, 8 to 15 mm, 8 to 12 mm, 8 to 10 mm, or 10 to 15 mm, for example, 10 to 12 mm, but is not limited thereto.

The length of the reaction tube may be preferably 270 to 720 mm, 270 to 630 mm, 270 to 540 mm, 360 to 810 mm, 360 to 720 mm, 360 to 630 mm, 360 to 540 mm, 450 to 810 mm, 450 to 720 mm, 450 to 630 mm, 450 to 540 mm, 540 to 810 mm, or 540 to 720 mm, for example, 540 to 630 mm, but is not limited thereto.

The present disclosure is directed to a method for producing an aromatic from a methane-containing mixture feed via a light hydrocarbon, and according to an embodiment of the present disclosure, an aromatic can be produced with a high yield and selectivity from a methane-containing mixture feed by a new method.

### BRIEF DESCRIPTION OF THE DRAWINGS

The above and other aspects, features and advantages of the present disclosure will be more apparent from the following detailed description taken in conjunction with the accompanying drawings.

FIG. 1 is a schematic diagram showing a scheme of the entire process of producing aromatics from methane according to an embodiment of the present disclosure.

FIG. 2 is a schematic diagram showing a low-temperature plasma reactor system according to an embodiment of the present disclosure.

FIG. 3A is a cross-sectional view of a multi-tubular reactor system based on low-temperature plasma reaction tubes according to an embodiment of the present disclosure.

FIG. 3B is a longitudinal sectional view of a multi-tubular reactor system based on low-temperature plasma reaction tubes according to an embodiment of the present disclosure.

FIG. 4 is a schematic diagram of a zeolite-based high-temperature catalyst reactor according to an embodiment of the present disclosure.

### DETAILED DESCRIPTION OF THE EXEMPLARY EMBODIMENTS

Hereinafter, the present disclosure may be modified in various forms and may have various embodiments, the following exemplary embodiments are illustrated in the accompanying drawings and are described in detail with reference to the drawings. However, it should be understood that the present disclosure is not intended to limit specific forms but intended to cover all the modifications, equivalents, or substitutions belonging to the idea and technical scope of the present disclosure.

Throughout the present specification, the “%” used to express the concentration of a specific material, unless otherwise particularly stated, refers to (wt/wt) % for solid/solid, (wt/vol) % for solid/liquid, and (vol/vol) % for liquid/liquid.

An aromatic producing process according to an embodiment of the present disclosure is largely attained by a low-temperature plasma reactor, a separator, and a zeolite-based high-temperature reactor, while pre-treatment unit(s) for treating a methane-containing feed and a post-treatment process for separating/purifying an aromatic mixture are not shown in the drawings. These pre-treatment unit(s) and post-treatment process can be applied by similar treatment unit(s) or processes that are employed in existing petrochemical and gas chemical processes.

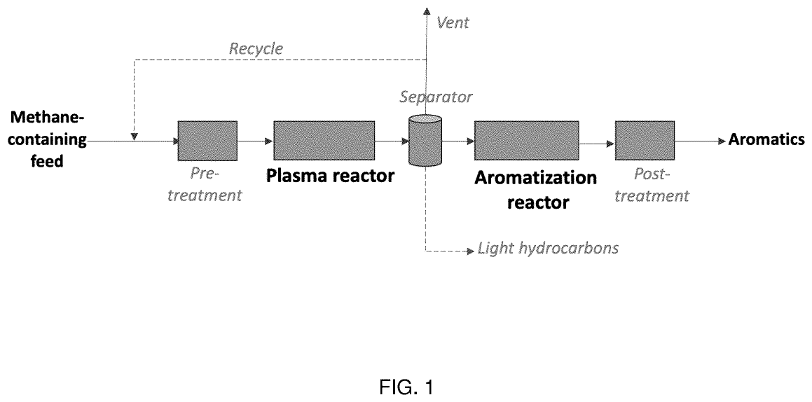

FIG. 1 is a block diagram showing a production process for synthesizing aromatics from a methane-containing mixture gas according to an embodiment of the disclosure.

Referring to FIG. 1, the present disclosure is directed to a process for producing aromatics from a methane-containing feed, via pre-treatment, a plasma reactor, a separator, an aromatization reactor, and a post-treatment, wherein unreacted methane is separated by the separator and is recycled either partially or entirely into a pre-treatment unit and, according to the need of the process, a portion of light hydrocarbon products (C2+hydrogen carbon compounds, etc.) are separated or removed.

Most (more than 70%) of C2 to C4 light hydrocarbons are transferred to the aromatization reactor. The light hydrocarbons are subjected to an aromatization reaction to obtain high value-added compounds, such as benzene, toluene, xylenes, ethylbenzene, and styrene, which are then separated and purified into desired products using a post-treatment unit.

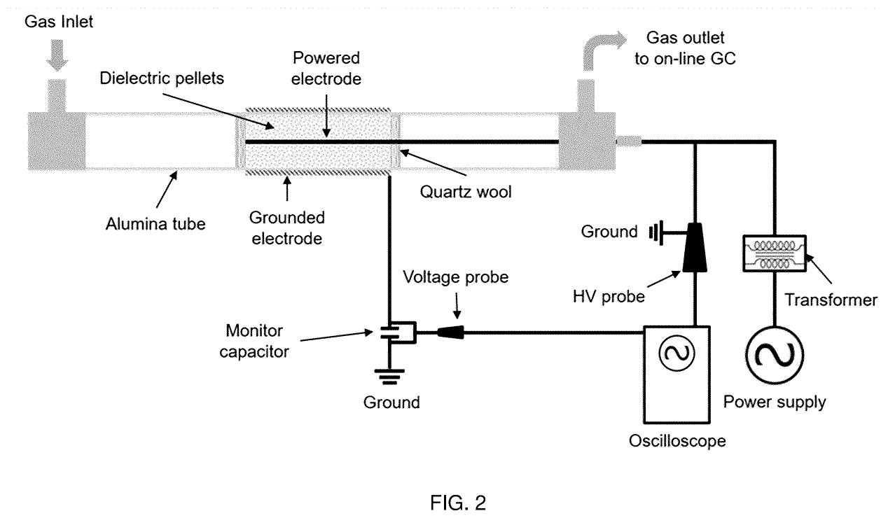

FIG. 2 is a schematic diagram showing a low-temperature plasma reactor system according to an embodiment of the present disclosure.

Referring to FIG. 2, the methane-containing mixture feed that has passed through the pre-treatment unit (in some cases, the pre-treatment unit can be omitted) is injected into the reactor where low-temperature plasma is generated, wherein a catalyst bed or a reaction bed may be filled with a catalyst of solid particles or only a reaction space in a non-catalytic state may be utilized. A high voltage (HV) electrode is installed inside a reaction tube, and a ground electrode and a capacitor are installed outside the reaction tube. In addition, a transformer and an AC power supply are installed toward the high voltage (HV) electrode. The members are configured like an electric circuit, as shown in the drawing, and an oscilloscope analyzer is installed to check the current, voltage, electrical charge, and the like inside the reaction tube. During the reaction, the outflowing mixture from the outlet can be analyzed using on-line gas chromatography (GC) to determine the type and amount of hydrocarbon compounds generated in real time.

A tubular reactor form is disclosed wherein the above-described members are provided for the tube and reactants and products flow into and out of the tube. However, as illustrated above, the form of the dielectric barrier reactor is not limited to the example described above. In addition to the above-described form, the dielectric barrier reactor may be provided in various forms, such as a multi-load reactor and a multi-stacked cell reactor. A specific description is additionally provided in examples.

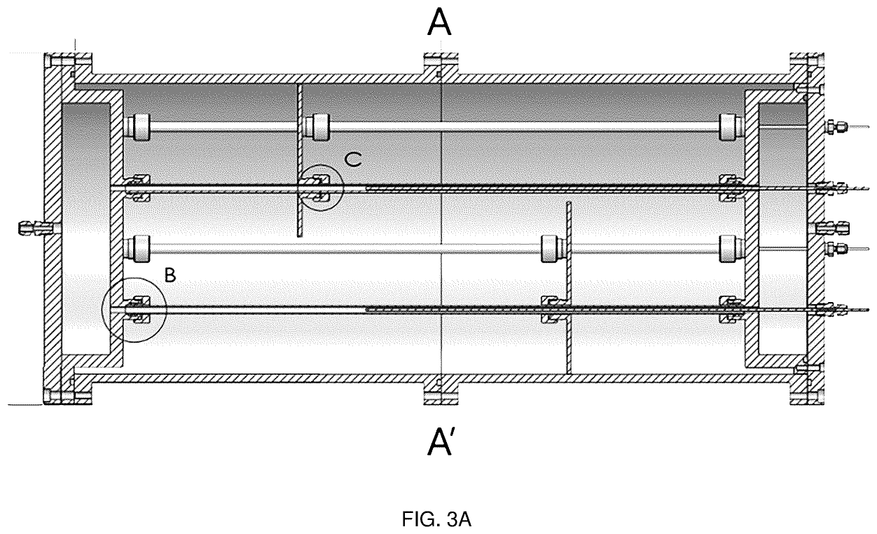

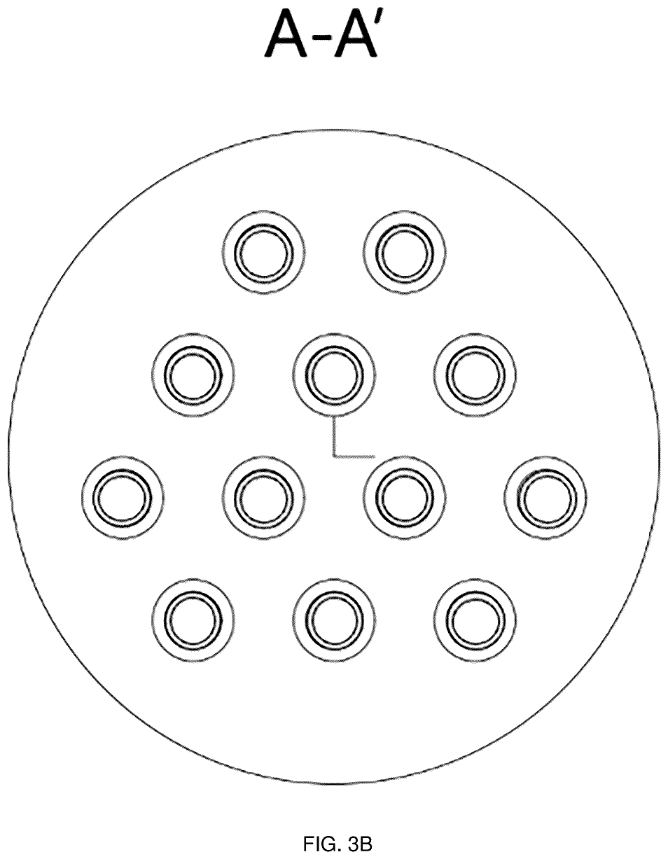

FIG. 3 shows a cross-sectional view and a longitudinal sectional view of a multi-tubular reactor system based on low-temperature plasma reaction tubes according to an embodiment of the present disclosure.

Referring to FIG. 3, a multi-tube reactor system with 12 low-temperature plasma reaction tubes is shown. The reactor, which can be confirmed in the cross-sectional view of FIG. 3A, is composed of reaction tubes, a shell space, two mixing sections (which are two spaces at the left and right ends of the reactor and can induce the mixing by filling the spaces with a filler or placing mixer devices in the spaces), and the like. The shell space may be filled with an inert gas or have an inert gas flow therethrough, and this feature can serve to adjust the temperature or prevent unnecessary oxidation in the reactor. Alternatively, the shell space may be filled with an insulation oil or have an insulation oil flow therein, and this feature can prevent unnecessary discharge or oxidation outside the reaction tubes, thereby efficiently use energy. FIG. 3B is a longitudinal sectional view taken along the line A-A′ indicated in FIG. 3A, showing that a plurality of tubes are installed.

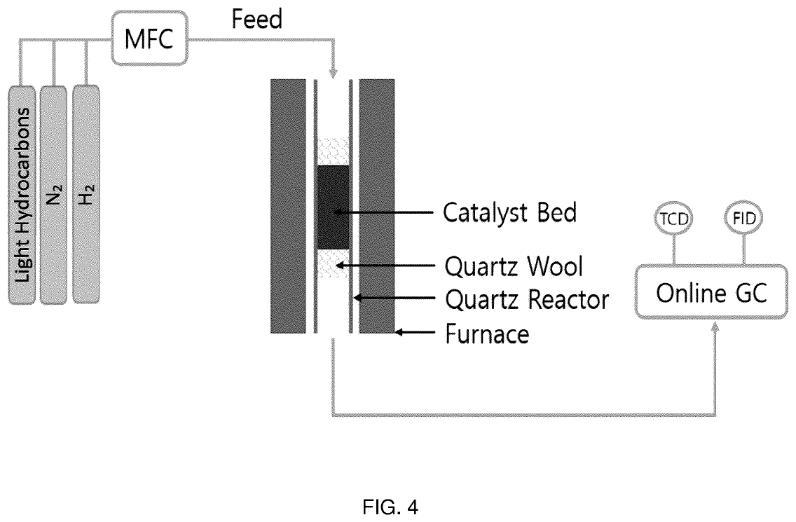

FIG. 4 is a schematic diagram of a zeolite-based high-temperature catalyst reactor according to an embodiment of the present disclosure.

Referring to FIG. 4, a reactor system is illustrated where light hydrocarbons (C2 to C4), hydrogen, and nitrogen are introduced and aromatized. The reactor system is composed of three types of feed cylinders, a mass flow controller (MFC, separately connected to a computer control system), a quartz reaction tube, a heating furnace, a catalyst bed, and an on-line GC (using a thermal conductivity detector (TCD) and a thermal flame ionization detector (FID)). A more specific description is provided through the following examples.

### Example 1: Performing Low-Temperature Plasma Reaction

A non-oxidative methane coupling reaction was conducted in a lab-made dielectric barrier plasma reactor system (FIG. 2) at atmospheric pressure and a temperature near room temperature.

The volumetric flow rate of a methane mixture (CH₄:N₂=1:1) was 40 sccm, and the total reaction time was 1,000 min. An alumina tube used as a dielectric barrier had an inner diameter of 6 mm and a thickness of 2 mm. A stainless steel rod with a diameter of 3 mm was used as a high-voltage electrode inside the reaction tube, and a steel wire as a ground electrode was wound on the outer wall. A discharge region by winding of the ground electrode was 150 mm in length. Therefore, in the reaction tube, the discharge gap was 1.5 mm, the discharge volume was 3.181 cm³, and the space velocity (SV) was 754.5 h⁻¹.

The plasma discharge region was completely filled with α-Al₂O₃(alumina) as a dielectric material. α-Al₂O₃ was prepared by heat-treating γ-Al₂O₃(Sigma-Aldrich, USA) at 1,000° C. for 8 hours, and three classifications of physical particle sizes were controlled using a sieve. According to the classified sizes, 0-53 μm: S, 53-100 μm: M, and 100-150 μm: L are designated.

A sinusoidal AC power supply (0-220 V, 60-1,000 Hz) was connected to a transformer (0-20 kV, 1,000 Hz), and through this electrical system, a high voltage was continuously applied to a plasma bed. The voltage and frequency applied to the plasma bed were fixed as 15 kV and 1 kHz, respectively. A capacitor with a capacitance of 1 μF was connected in series between the plasma bed and the ground. The voltage applied to the plasma bed was measured using a high-voltage probe (1,000:1, P6015A, Tektronix). The voltage of the 1-ρF capacitor was measured using a voltage probe (10:1, P6100, Tektronix) connected to each side of the capacitor. A current probe (TCP202, Tektronix) is connected to the ground electrode to evaluate the current profile across the DBD plasma bed. The probe was connected to a digital oscilloscope (TDS 3012C, Tektronix). The quantity of electricity accumulated in the plasma bed was calculated by multiplying the voltage across the capacitor and the capacitance (1 ρF) of the capacitor. The discharge power applied to the bed during the reaction was calculated to be 44.1 W.

During the reaction, the temperatures of the reactor were measured by an IR temperature detector. The temperature at the inlet was almost room temperature, and the bed temperature was monitored. The highest temperature was observed to be 100° C. in the central region of the reactor. The temperatures observed in the other regions were 100° C. or lower, and were mostly close to room temperature. An external insulation or an oven was not used in this reactor system.

The outflowing gas from the plasma bed was measured by gas chromatography (6500GC Young Lin Instrument Co., Korea) using a Porapak-N and Molecular Sieve 13× column connected to a thermal conductivity detector (TCD) and a GasPro column connected to a flame ionization detector (FID). H₂, N₂, and CH₄ in the outflowing gas were detected using a TCD. CH₄, C₂H₂, C₂H₄, C₂H₆, C₃H₆, C₃H₈, 1-C₄H₈, and n-C₄H₁₀ in the outflowing gas were detected using an FID.

TABLE 1

| | Conv. (%) | Yield (mol %) | | | | | | |
|---|---|---|---|---|---|---|---|---|
| Composition | CH₄ | H₂ | C₂H₂ | C₂H₄ | C₂H₆ | C3 HCs | C4 HCs | C2-C4 |
| Blank | 18.87 | 7.92 | 0.51 | 0.47 | 6.22 | 3.00 | 2.46 | 12.66 |
| S | 54.83 | 31.2 | 25.5 | 3.84 | 5.70 | 4.10 | 13.8 | 52.94 |
| M | 58.36 | 33.38 | 15.24 | 3.17 | 7.10 | 4.16 | 11.38 | 41.05 |
| L | 46.38 | 23.33 | 9.45 | 3.29 | 10.12 | 4.98 | 5.22 | 33.06 |

As shown in Table 1, the bed filled with alpha phase (α) alumina showed higher conversion rates in all the cases than in the blank test during the initial reaction due to the high electric field strength between the dielectric particles. Additionally, the selectivity for C2 compounds, such as ethylene (C₂H₄) and acetylene (C₂H₂), was much higher in the filled bed tests than in the blank test. In contrast, the selectivity for ethane (C₂H₆) in the blank test was higher than that in the filled bed test. In terms of yield, unsaturated C2 compounds were produced in higher quantities in the filled bed tests than in the blank test. The use of the medium-sized particles (M size) resulted in highest conversion rates, and the use of the smallest-sized particles (S size) showed a highest selectivity for C₂H₂ among the C2 compounds.

The yields of all the hydrocarbon products were calculated on a c-mol basis, reflecting that two hydrogen molecules can be generated per methane in conversion. A considerable amount of coke was produced due to the high initial activity, while a small number of carbon balances were measured to be below 100%.

### Example 2: Performing Mixture Gas Conversion Reaction Utilizing Zeolite Catalyst (1)

On the basis of the experimental results using the M catalyst showing the highest conversion performance in Example 1, the feed composition into the zeolite-based high-temperature reactor was established as shown in Table 2 below.

TABLE 2

| | | | | | | | C3 | C4 | C₅₊ HCs | |
|---|---|---|---|---|---|---|---|---|---|---|
| Composition | CH₄ | N₂ | H₂ | C₂H₂ | C₂H₄ | C₂H₆ | HCs | HCs | & Others | total |
| Products from low- | 19.6 | 43.4 | 27.2 | 5.5 | 0.8 | 1.2 | 0.6 | 1.5 | 0.2 | 100 |
| temperature | | | | | | | | | | |
| plasma reactor | | | | | | | | | | |
| (mol %) | | | | | | | | | | |
| Feeds into zeolite- | — | 50 | 36.84 | 7.51 | 1.13 | 1.68 | 0.81 | 2.04 | — | 100 |
| based high- | | | | | | | | | | |
| temperature reactor | | | | | | | | | | |
| (mol %) | | | | | | | | | | |

The feed composition was determined by assuming a process scheme of using a feed formed by separating and recycling methane from all the products in Example 1, supplying C2 to C4 light hydrocarbons to the zeolite-based high-temperature as shown in FIG. 4, with C5 or higher hydrocarbons separated through a separation process, and adding 50 mol % nitrogen in order to achieve an appropriate concentration of a reactant. Particularly, the conversion rates, selectivities, and yields for hydrocarbons were on a c-mol basis as in Example 1.

HZSM-5 catalyst was used as the zeolite catalyst. HZSM-5 (11.5) was synthesized by the following method. Ammonium-form ZSM-5 (11.5) catalyst (Zeolyst, USA) with a Si/Al ratio=11.5 was calcined in a muffle furnace at 550° C. for 6 hours, while high-purity air was injected at a flow rate of 200 mL/min, thereby synthesizing H-form HZSM-5 (11.5) catalyst.

The reaction was carried out at 700° C. and atmospheric pressure by using a mixture gas as a reactant, with a space velocity of 20,000 mL/g_{cat}⁻¹h⁻¹. An aromatization reaction of the mixture gas was carried out in a lab-made immobilized bed reactor system, which is thermochemical microsystem, as shown in FIG. 4. This reactor was fabricated of a quartz tube with an inner diameter of 10 mm and a length of 540 mm, and a porous silica disk was
used as a spacer, on which the HZSM-5 (11.5) catalyst was filled.

The outflowing gas from the reactor was measured by a gas chromatography (6500GC Young Lin Instrument Co., Korea) using a Carboxen-1000 column connected to a thermal conductivity detector (TCD) and a GasPro column connected to a flame ionization detector (FID). H₂, N₂, and CH₄ in the outflowing gas were detected using the TCD. CH₄, C₂H₂, C₂H₆, C₂H₄, C₃H₈, n-C₄H₈, benzene, toluene, xylenes (ortho, meta, para) and the like in the outflowing gas were detected through the FID.

TABLE 3

| | Aromatization reaction products | | | | | | | | | Paraffin, |
|---|---|---|---|---|---|---|---|---|---|---|
| | | | | | | C9 | | | Total | carbon, |
| Composition | Benzene | Toluene | Xylenes | Ethylbenzene | Styrene | Aromatics | Naphthalene | BTX | aromatics | etc. |
| Yield | 28.23 | 11.64 | 1.09 | 0.06 | 0.28 | 3.78 | 11.15 | 40.96 | 56.23 | 43.77 |
| (carbon- | | | | | | | | | | |
| mol %) | | | | | | | | | | |

As a result of the conversion of the mixture gas through the use of HZSM-5 (11.5) as a catalyst, as shown in Table 3, the selectivity for benzene was 35.17%, the selectivity for toluene was 14.50%, the selectivity for xylenes was 1.35%, the selectivity for BTX was 51.02%, and the selectivity for total aromatics was 70.05%, the yield of BTX was 40.96%, and the yield of total aromatics was 56.23%, at a time on stream (TOS) of 15 min. These results confirmed that the mixture gas was well converted into BTX and aromatics. It can be therefore seen that aromatics, such as naphthalene as well as benzene, toluene, and xylenes, were well synthesized. It can be also seen that the HZSM-5 (11.5) was suitable for an aromatization reaction of the mixture gas.

Considering the flow rates and the amounts of products used in the experiment, the volume of the reactor required as the zeolite-based high-temperature reactor was about ½ that of the low-temperature plasma reactor.

The results of conversion of the mixture gas through the use of HZSM-5 (11.5) as a catalyst at TOS 15 min are shown in Table 4. The yield of BTX and the yield of total aromatics from methane were 21.7% and 29.8%, respectively, as shown in Table 4. The yields of BTX and total aromatics from methane were calculated using the following equations. The terms of each equation were calculated on a c-mol basis.

$$Yield of BTX or total aromatics from methane = Yield of C 2 to C 4 in first reaction \times Yield of BTX or total aromatics in second reaction = \frac{output molar flow rate of C 2 — C 4 hydrocarbons}{input molar flow rate of CH 4} \times \frac{output molar flow rate of BTX or total aromatics}{input molar flow rate of C 2 — C 4 hydrocarbons}$$

TABLE 4

| | Yield |
|---|---|
| | (carbon-mol %) |
| Yield of BTX from methane through plasma reactor | 21.7 |
| Yield of total aromatics from methane through plasma | 29.8 |
| reactor | |

It can be therefore confirmed that methane was well converted into BTX and aromatics via the mixture gas. It can be therefore seen that aromatics, such as naphthalene as well as benzene, toluene, and xylenes, were well synthesized.

### Example 3: Performing Mixture Gas Conversion Reaction Utilizing Zeolite Catalyst (2)

To investigate the reproducibility of the zeolite-based high-temperature reactor tested in Example 2 above, the experiment was repeated under the same conditions. On the basis of the experimental results using the M catalyst showing the highest conversion performance in Example 1, the feed composition into the zeolite-based high-temperature reactor and the conversion rates, selectivities, and yields of hydrocarbons were calculated as shown in Table 2 below. The other conditions for the experiment, along with the type of catalyst and the reactor operation method are the same as those in Example 2.

TABLE 5

| | Aromatization reaction products | | | | | | | | | Paraffin, |
|---|---|---|---|---|---|---|---|---|---|---|
| | | | | | | C9 | | | Total | carbon, |
| Composition | Benzene | Toluene | Xylenes | Ethylbenzene | Styrene | Aromatics | Naphthalene | BTX | aromatics | etc. |
| Yield | 25.68 | 12.84 | 1.05 | 0.08 | 0.33 | 5.89 | 15.61 | 39.58 | 61.48 | 38.52 |
| (carbon- | | | | | | | | | | |
| mol %) | | | | | | | | | | |

As shown in Table 5, the selectivity for benzene was 32.05%, the selectivity for toluene was 16.03%, the selectivity for xylenes was 1.31%, the selectivity for BTX was 49.39%, and the selectivity for total aromatics was 76.71%, the yield for BTX was 39.58%, and the yield for total aromatics was 61.48%, at a time on stream (TOS) of 55 min. These results confirmed that the mixture gas was well converted into BTX and aromatics. It can be therefore seen that aromatics, such as naphthalene as well as benzene, toluene, and xylene, were well synthesized. The reproducibility in results could be confirmed as in Example 2 above, and it can be seen that HZSM-5 (11.5) is suitable for an aromatization reaction of the mixture gas.

The results of conversion of the mixture gas through the use of HZSM-5 (11.5) as a catalyst at TOS 55 min are shown in Table 6.

TABLE 6

| | Yield |
|---|---|
| | (carbon-mol %) |
| Yield of BTX from methane through plasma reactor | 20.9 |
| Yield of total aromatics from methane through plasma | 32.6 |
| reactor | |

The yield of BTX and the yield of total aromatics from methane were 20.9% and 32.6%, respectively, as shown in Table 6. It can be therefore confirmed that methane was well converted into BTX and aromatics via the mixture gas. It can be therefore seen that aromatics, such as naphthalene as well as benzene, toluene, and xylenes, were well synthesized. These results were confirmed to be reproducible, as demonstrated in Example 2.

What is claimed is:

1. A method for producing aromatics from a methane-containing mixture, the method comprising:
a first reaction step of converting a methane-containing mixture, which is supplied, into C2 to C4 light hydrocarbons by inducing a low-temperature plasma reaction in the methane-containing mixture; and
a second reaction step of synthesizing aromatics from a substrate containing the products in the first reaction step, in the presence of a zeolite catalyst.

2. The method of claim 1, wherein the first reaction step is performed by generating plasma through the supply of an alternating current power at an applied voltage of 10 to 20 kV and a frequency of 0.1 to 5 kHz.

3. The method of claim 1, wherein the first reaction step is performed in a temperature condition of 25 to 100° C.

4. The method of claim 1, further comprising a separation step of adjusting the amount or composition of a reactant, which is generated in the first reaction step and supplied for performing the second reaction step.

5. The method of claim 1, wherein the methane-containing mixture contains methane and nitrogen at a volume ratio of 8:2 to 2:8.

6. The method of claim 1, wherein the second reaction step is performed by using, as a substrate, C2 to C4 light hydrocarbons, nitrogen, and hydrogen.

7. The method of claim 6, wherein the substrate is a mixture gas containing 2 to 15 mol % of C₂H₂, 0.2 to 8 mol % of C₂H₄, 0.2 to 8 mol % of C₂H₆, 0.2 to 8 mol % of a C3 hydrocarbon, 0.5 to 12 mol % of a C4 hydrocarbon, 40 to 60 mol % of N₂, and 30 to 45 mol % of H₂.

8. The method of claim 1, wherein the second reaction step is performed under conditions of a temperature of 500 to 800° C. and a space velocity of 10,000 to 30,000 mL/g_{cat}⁻¹h⁻¹.

9. The method of claim 1, wherein the zeolite catalyst has a Si/Al ratio of 6 to 17.

10. The method of claim 1, wherein the aromatics are one or more selected from the group consisting of benzene, toluene, xylenes, ethyl benzene, and styrene.

11. An apparatus for producing aromatics from a methane-containing mixture, the apparatus comprising:
a first reaction unit comprising a low-temperature plasma reactor including a reaction tube where a low-temperature plasma is generated; and
a second reaction unit comprising a zeolite-based high-temperature reactor: including a reaction tube having a zeolite catalyst bed therein; and a furnace adjacent to the reaction tube.

12. The apparatus of claim 11, wherein the low-temperature plasma reactor is a multi-tubular reactor having two or more reaction tubes.

13. The apparatus of claim 11, further comprising a separator configured to adjust the amount or composition of an inflowing material, which is generated in the first reaction unit and supplied for the performance of the second reaction unit.
